(19) United States
(12) Patent Application Publication
(10) Pub. No.: US 2025/0354844 A1
(43) Pub. Date: Nov. 20, 2025

(54) MICROFLUIDIC ELECTROCHEMICAL DEVICE FOR MEASURING A VOLUME FLOW RATE

(71) Applicant: NOPTRACK, Castres (FR)

(72) Inventors: Fethi BEDIOUI, Paris (FR); Thomas DELAHAYE, Mondeville (FR); Sophie GRIVEAU, Massy (FR); Catherine SELLA, Meudon la forêt (FR); Laurent THOUIN, Antony (FR); Christian AMATORE, Paris (FR); Gilles FAVRE, Cugnaux (FR)

(21) Appl. No.: 18/874,036

(22) PCT Filed: Jun. 26, 2023
(86) PCT No.: PCT/EP2023/067355

§ 371 Date: Dec. 11, 2024

(30) Foreign Application Priority Data
Jun. 29, 2022 (FR) FR2206577

(51) Int. Cl. G01F 1/64; G01F 1/704; G01F 1/7088

(52) U.S. Cl. CPC G01F 1/64; G01F 1/7046; G01F 1/7088

(57) ABSTRACT

A microfluidic electrochemical device has a microfluidic channel and an electrochemical cell having a pair of working electrodes separated by an inter-electrode distance in a flow direction of the fluid in the microfluidic channel, a counter-electrode and a reference electrode. The microfluidic electrochemical device has an electrochemical amperometry measurement system configured to bias the pair of working electrodes so that each electrode produces an amperometric signal by oxidation reaction or by reduction reaction with the electroreactive fluid or with a chemical species associated with a redox couple intended for the fluid. The microfluidic electrochemical device determines the volume flow rate of the fluid in the microfluidic channel, notably based on the inter-electrode distance and a time delay between the amperometric signals produced by the pair of working electrodes.

### TECHNICAL FIELD

The invention relates to the field of microfluidic electrochemical devices and to methods for measuring a volume flow rate of a fluid that is electroactive or contains one or more electroactive species in a microfluidic channel. More generally, the invention also relates to an apparatus for determining a quantitative sweating parameter of a human or animal subject.

### TECHNOLOGICAL BACKGROUND

Sweat is secreted by the sweat glands of the skin, discharged through the pores of the skin and evaporated from the epidermis. Sweating plays an important role in the organism of the subject, since it enables the body to regulate temperature through perspiration. Excessive sweating can lead to the dehydration of the subject, impair their physical performance capabilities and have harmful consequences on their health. Similarly, excessive water consumption can lead to hyponatremia, fatigue, confusion, coma and even the death of the subject.

Various devices for measuring micro-flow rates in situ are known in the prior art, notably thermal flow sensors or even Coriolis effect micro-flowmeters. In practice, these devices are expensive, exhibit unresolved reliability issues and are generally incorporated into bulky units. More specifically, microfluidic devices have been developed to measure micro-flow rates of sweat perspired by a human or animal subject that circulates in microfluidic channels. Measuring a sweat micro-flow rate allows a quantitative sweating parameter of the subject to be assessed in order, for example, to monitor the hydration level of the subject in order to prevent water imbalances in the body, particularly in athletes and the elderly, or even to diagnose hypohidrosis, which is a sweating disorder characterized by insufficient sweat production and which can be caused by pathologies that are likely to damage the functioning of the sweat glands (diabetes, alcoholism, Parkinson's disease, Ross's syndrome, Sjögren's syndrome, small-cell lung cancer, etc.), by cutaneous causes (burns, inflammations, infections, skin pathologies, etc.), by medicinal causes (for example, anticholinergic treatments) and genetic causes (for example, hypohidrotic ectodermal dysplasia).

Known microfluidic devices have the advantage of being able to easily collect sweat and without evaporating the sweat in microfluidic channels with very high temporal resolution. Colorimetric techniques are generally preferred due to the ease of manufacturing the associated devices. However, their main disadvantage relates to the irreversible nature of the method. Once the microfluidic channel has been filled with sweat, these devices are permanently modified by the dyes and can no longer be used. This situation is comparable to detection techniques that are based on electrical transduction signals such as resistance, conductance, capacitance or impedance. In order to estimate sweat micro-flow rates, their implementation involves monitoring the filling rate of the channels by means of electrodes disposed along the microfluidic channels. Once filled, the microfluidic channels can no longer be used.

Document FR 3103901 A1 notably describes a method for measuring a flow speed of sweat that is based on a delay between the time variations in amperometric signals that intrinsically represent the concentration of hydrogen peroxide H₂O₂, nitric oxide NO or nitrite ion NO₂⁻, present in the flow of sweat.

### SUMMARY

One idea behind the invention is to provide a microfluidic electrochemical device for measuring the volume flow rate of a fluid in a microfluidic channel, without necessarily determining the concentration of electroactive chemical species contained in the flow of fluid.

Another idea behind the invention is to provide a flexible device to be adhered to the skin for determining a quantitative sweating parameter of a subject based on the continuous in situ measurement of the volume flow rate of sweat flowing in a microfluidic channel.

One aim of the invention is to provide such a device with the additional advantages of being space-saving, of having a simple design and of being low cost.

According to one embodiment, the invention provides a microfluidic electrochemical device for measuring a flow speed and/or a volume flow rate of a fluid, the fluid comprising a solvent, the microfluidic electrochemical device comprising:
at least one microfluidic channel configured to allow the fluid to flow in a flow direction;
at least one electrochemical cell disposed in the at least one microfluidic channel, the electrochemical cell comprising a first working electrode and at least one second working electrode spaced apart from the first working electrode by an inter-electrode distance in the flow direction, at least one counter-electrode and at least one reference electrode; and
an electrochemical amperometry measurement system configured to bias the first working electrode at a first electrode potential and the second working electrode at a second electrode potential, so that each of said first and second working electrodes produces an amperometric signal by oxidation reaction or by reduction reaction of the solvent or with at least one chemical species forming a redox couple with the solvent;
the electrochemical amperometry measurement system being configured to determine the flow speed and/or the volume flow rate of the fluid in the microfluidic channel based on the inter-electrode distance between the first and second working electrodes, and a time delay between a variation in the amperometric signal produced by the first working electrode and a variation in the amperometric signal produced by the second working electrode.

Such a microfluidic electrochemical device can be incorporated into numerous microsystems for in situ measurement of the volume flow rate and/or the flow speed of a fluid in a microfluidic channel. These microsystems can be, for example, lab-on-a-chip microfluidic platforms or micro-Total Analysis Systems (μTAS).

The microfluidic electrochemical device is simple to manufacture and easy to industrialize, as there are no moving parts and no need to make any assumptions concerning the hydrodynamic regime of the flow of the fluid in the microfluidic channel. Determining the flow speed and/or the volume flow rate is in no way linked to determining the concentration of chemical species generated or contained in the fluid, but solely to a response time between variations in the amperometric signals of a pair of working electrodes.

According to some embodiments, such a microfluidic electrochemical device can comprise one or more of the following features.

According to one embodiment, the solvent is water H₂O₍ₗ₎.

Water can act as a reducing chemical species in the O₂/H₂O redox couple and as an oxidizing chemical species in the H₃O⁺/H₂ redox couple.

According to one embodiment, the fluid is sweat from a human or animal subject.

According to one embodiment, the first electrode potential allows the oxidation of water H₂O₍ₗ₎ to dioxygen O_{2(aq)} and the second electrode potential allows the reduction of the dioxygen O₂ dissolved in the produced water H₂O to water H₂O.

Selecting the oxidation reactions of the water H₂O₍ₗ₎ at the first working electrode and the reduction of the dioxygen O_{2(aq)} at the second working electrode, by virtue of the appropriate potentials applied to the first and second working electrodes, allows the amplitude of the amperometric signals detected on each of said working electrodes to be controlled without necessarily measuring these amplitudes, but in such a way as to maintain a sufficient signal-to-noise ratio to allow easy detection of variations in the amperometric signals.

According to one embodiment, the first electrode potential allows the reduction of water H₂O₍ₗ₎ to dihydrogen H_{2(aq)} and the second electrode potential allows the reduction of water H₂O₍ₗ₎ to dihydrogen H_{2(aq)}.

According to one embodiment, the first electrode potential allows the reduction of dioxygen O_{2(aq)} dissolved in water H₂O₍ₗ₎ to water H₂O₍ₗ₎ and the second electrode potential allows the reduction of dioxygen O_{2(aq)} dissolved in water H₂O₍ₗ₎ to water H₂O₍ₗ₎.

According to one embodiment, the electrochemical amperometry measurement system is also configured to:
during a first step, bias the first working electrode at the first electrode potential and the second working electrode at the second electrode potential;
during a second step, disconnect the first working electrode or set the first electrode potential at a potential close to or equal to a zero-current equilibrium potential.

According to one embodiment, the microfluidic electrochemical device further comprises an isolating support, with said at least one microfluidic channel being formed in the isolating support, the first and second working electrodes being formed by metal deposits of platinum or platinum black on said isolating support.

According to one embodiment, the counter-electrode is positioned downstream of the working electrodes in the flow direction, and wherein the reference electrode is positioned upstream of said working electrodes in said flow direction.

Thus, the reference electrode is located upstream of the pair of working electrodes in order to maintain the stability of the reference electrode potential over time; and the counter-electrode is located downstream of the pair of working electrodes and, therefore, from the reference electrode, so that the chemical species generated on its surface disrupt neither the working electrodes nor the reference electrode.

Advantageously, the surface area of the counter-electrode is two to three times greater than that of the other electrodes.

According to some embodiments, the microfluidic electrochemical device can comprise one or more microfluidic channels. If applicable, an electrochemical cell can be disposed in one or each microfluidic channel or in some or all of the microfluidic channels. The electrochemical cells disposed in various channels can be different or identical. The redox reactions implemented in the electrochemical cells disposed in various channels can be different or identical.

According to one embodiment, the microfluidic electrochemical device comprises a first and a second microfluidic channel, with the first, respectively, the second, electrochemical cell being disposed in the first, respectively, the second, microfluidic channel, with the inter-electrode distance of the first electrochemical cell being different from the inter-electrode distance of the second electrochemical cell.

According to one embodiment, said at least one electrochemical cell comprises two second working electrodes respectively separated from the first working electrode by a first inter-electrode distance and by a second inter-electrode distance, with the first inter-electrode distance being different from the second inter-electrode distance.

Preferably, the inter-electrode distance separating the pair of working electrodes is selected so as to be small enough for changes in the physiological response of the subject to be negligible for the duration of the time delay between the variations in the amperometric signals of the working electrodes, and large enough to allow, at least in one of the microfluidic channels, a decoupled operating regime for the working electrodes.

Indeed, the coupled or decoupled operating regime of the working electrodes depends on the average flow speed of the fluid in the microfluidic channel and on the inter-electrode distance. Δt high flow speeds, if the inter-electrode distance is too small, the flow of fluid that has reacted at the first working electrode is still inhomogeneous after reaching the second working electrode. This coupling regime limits the temporal resolution of the amperometric signals, which degrades the accuracy of the sweat volume flow rate measurements.

According to one embodiment, the electrochemical amperometry measurement system is configured to determine the volume flow rate as a function of a cross-sectional surface area of said microfluidic channel in the flow direction.

By configuring the inter-electrode distance differently depending on the considered electrochemical cell, it is thus possible to measure a volume flow rate over a range of values covering all conceivable physiological flow rates.

According to one embodiment, the invention provides an apparatus intended to be placed on an investigation zone of an epidermis of a human or animal subject in order to measure a quantitative sweating parameter of the subject, said apparatus comprising:
a structure defining a microfluidic electrochemical device, the structure comprising an inlet orifice defining the investigation zone and allowing through sweat from the epidermis, the at least one microfluidic channel of the microfluidic electrochemical device being connected to the inlet orifice; and
an electronic processing device configured to determine the quantitative sweating parameter of said human or animal subject based on measurements of the volume flow rate of sweat carried out by the microfluidic electrochemical device.

The quantitative sweating parameter can be a sweating rate that is determined based on the total volume of sweat perspired by the subject over a given time range, in relation to the surface area of the investigation zone.

According to one embodiment, the quantitative sweating parameter of said human or animal subject is a sweating rate.

“Epidermis” is understood to mean the surface layer of the skin in humans and animals.

According to some embodiments, such an apparatus can comprise one or more of the following features.

According to one embodiment, the structure is a multi-layer structure comprising a lower layer and at least one layer superimposed on the lower layer, with the microfluidic electrochemical device extending parallel to the lower layer, the lower layer comprising said inlet orifice.

According to one embodiment, the multi-layer structure further comprises an upper layer and at least one intermediate layer located between the lower layer and the upper layer, with the microfluidic electrochemical device being formed within the thickness of the at least one intermediate layer.

The layers can be attached to each other using any suitable method, for example, by adhesives, by soldering, by mechanical clamping etc.

By virtue of these features, manufacturing, assembling and therefore industrializing the device is facilitated.

By virtue of these features, the apparatus is adapted to possible curvatures when applied to the epidermis. Furthermore, the one or more intermediate layers also allow a thickness to be created in order to compensate for the thickness of the electrodes of the microfluidic electrochemical device. This ensures that the apparatus is watertight.

According to one embodiment, the upper layer comprises an outlet orifice passing through the upper layer, and wherein the at least one microfluidic channel is connected to the outlet orifice.

According to one embodiment, the first working electrode, the at least one second working electrode, the at least one counter-electrode and the at least one reference electrode are disposed on an inner face of the upper layer closing the at least one microfluidic channel from above and/or are disposed on an upper face of the lower layer closing said at least one microfluidic channel from below.

By virtue of these features, the electrodes are disposed in a reliable manner. Furthermore, manufacturing the multilayer structure comprising these electrodes is facilitated in that the electrodes can be manufactured on a flat layer when the microfluidic electrochemical device is formed in an intermediate layer.

According to one embodiment, the apparatus further comprises a wired or wireless communication device configured to transmit one or more measurement signals produced by the microfluidic electrochemical device.

According to one embodiment, the apparatus further comprises a gyroscopic module and/or at least one accelerometer for detecting a state of activity of said human or animal subject.

According to one embodiment, the apparatus further comprises a temperature sensor configured to measure the temperature of the epidermis of said human or animal subject. According to one embodiment, the apparatus comprises a geolocation module.

By virtue of these features, the apparatus is configured to periodically carry out and transmit measurements, for example, at a configurable frequency or at a frequency that depends on a state of activity detected by the apparatus in order to facilitate an analysis of the correlations between the state of activity of the subject and the quantitative sweating parameter measured by the apparatus.

The measurements of the volume flow rate of sweat and/or of the quantitative sweating parameter can be used in various applications, for example, in order to monitor the hydration level of the subject in order to prevent body water imbalances, notably in athletes during exercise or in the elderly, particularly in hot weather, or even in order to diagnose hypohidrosis, irrespective of the cause.

Further applications are possible in various technological or environmental fields whenever the measurement of a flow rate is necessary in a device or a method involving an electroactive fluid or containing one or more electroactive species.

### BRIEF DESCRIPTION OF THE FIGURES

The invention will be better understood, and further aims, details, features and advantages thereof will become more clearly apparent throughout the following description of several particular embodiments of the invention, which are provided solely by way of a non-limiting illustration, with reference to the appended drawings.

FIG. 1 is a schematic back view of a subject on which an apparatus according to one embodiment has been placed;

FIG. 2 is a perspective view partially showing a multilayer structure for an apparatus according to one embodiment;

FIG. 3 is a cross-sectional view along line III-III of FIG. 2;

FIG. 4 is an exploded view of the multilayer structure according to one embodiment;

FIG. 5 is a partial functional schematic representation of a multilayer structure defining an electrochemical device in an apparatus;

FIG. 6 is a partial functional schematic representation of a microfluidic electrochemical device that can be used in an apparatus;

FIG. 7 is a schematic top view of an electrochemical cell according to a first embodiment;

FIG. 8 is a schematic view similar to that of FIG. 7, according to a second embodiment;

FIG. 9 is a functional schematic cross-sectional representation of an electrochemical cell along a microfluidic channel according to a first embodiment;

FIG. 10 is a set of chronoamperograms illustrating a method that can be implemented, according to the first embodiment, with the microfluidic electrochemical device of FIG. 7;

FIG. 11 is a functional schematic representation similar to that of FIG. 9 according to a second embodiment;

FIG. 12 is a set of chronoamperograms similar to those of FIG. 10 according to the second embodiment;

FIG. 13 is a functional schematic representation similar to those of FIGS. 9 and 11, according to a third embodiment;

FIG. 14 is a set of chronoamperograms similar to those in FIGS. 10 and 12, according to a third embodiment;

FIG. 15 is a schematic functional representation of an electronic control device that can be implemented with the apparatus.

### DESCRIPTION OF THE EMBODIMENTS

The embodiments described hereafter relate to an apparatus for determining a quantitative sweating parameter of a subject by means of a microfluidic electrochemical device for continuously measuring the volume flow rate of sweat in a microfluidic channel. More generally, such a microfluidic electrochemical device can be incorporated into numerous microsystems for the in situ measurement of the average flow speed of an electroactive fluid in a microfluidic channel. These microsystems can be, for example, lab-on-a-chip microfluidic platforms or micro-Total Analysis Systems (μTAS).

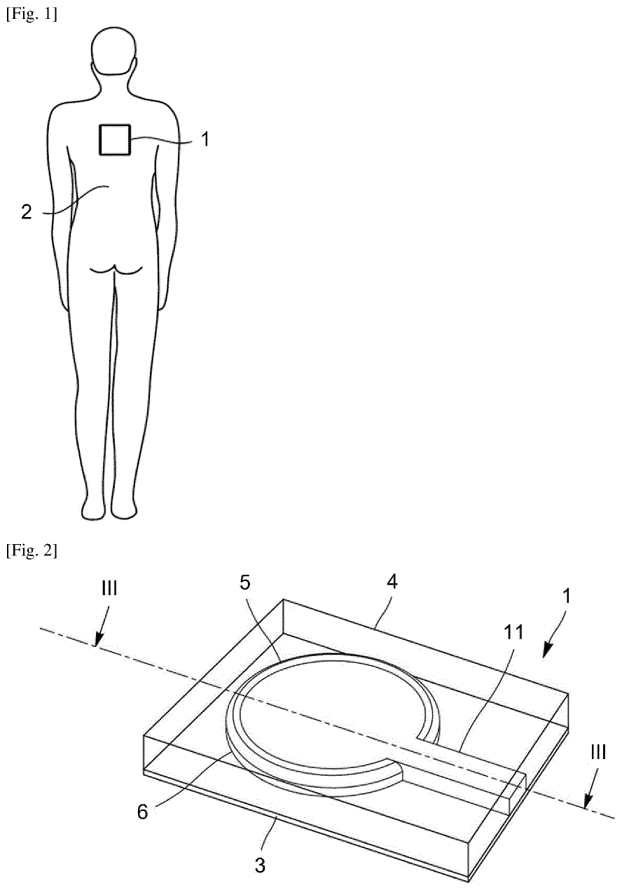

With reference to FIG. 1, the apparatus 1 for determining a quantitative sweating parameter is disposed on the skin 2 of a human subject, for example, on their back. In a variant, not shown, the apparatus 1 can be disposed on the skin of an animal subject.

With reference to FIG. 2, the apparatus 1 is, for example, in the form of a space-saving multilayer structure made of watertight materials, for example, a polymer material. The multilayer structure comprises a lower layer 3 made of a flexible and biocompatible material, preferably self-adhesive, for example, polyethylene terephthalate (PET), which can be positioned directly on the skin 2 of the subject, and an isolating support 4 superimposed on the lower layer 3.

A microfluidic channel 11 is hollowed out of the thickness of the isolating support 4. A sampling cup 5, located in line with a circular opening 6, is made in the lower layer 3.

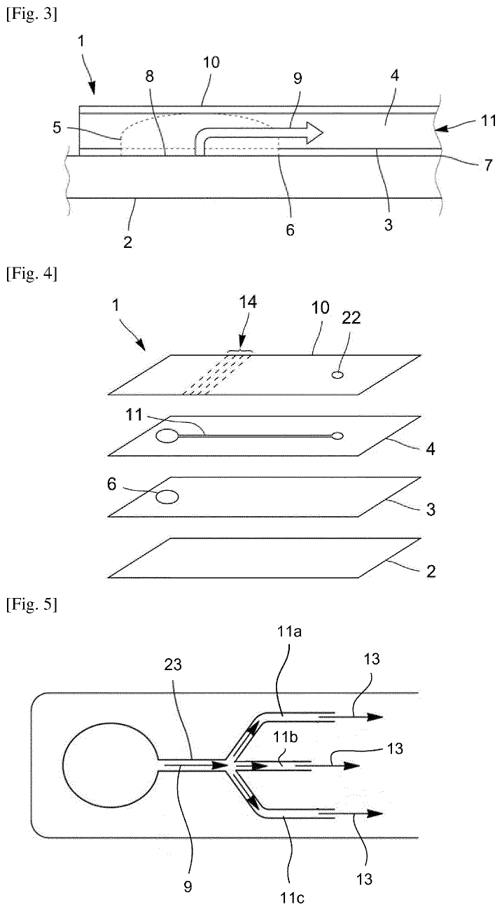

With reference to FIG. 3, the lower layer 3 is adhered to the skin 2 by an adhesive layer 7. A central part of the lower layer 3 and of the adhesive layer 7 comprises the circular opening 6 delimiting an investigation zone 8 on the skin 2 of the subject, for example, with a diameter of a few millimetres to a few centimetres. The circular opening 6 can assume any other shape, for example, an ellipse, a triangle, a rectangle, a square, a polygon or the like. The circular opening 6 forms an inlet orifice 6 for guiding a flow of sweat 9, notably in order to convey the sweat into the microfluidic channel 11. The flow of sweat 9 passes from the skin 2 of the subject through the circular opening 6 into the microfluidic channel 11.

A hydrophilic collection element (not shown), for example, a fibrous body such as cotton or a non-woven material, can be disposed in the circular opening 6 and the sampling cup 5. The collection element conveys the sweat produced in the investigation zone 8 to the microfluidic electrochemical device 100.

According to a first embodiment, with reference to FIG. 4, the multilayer structure of the apparatus 1 comprises a lower layer 3 comprising an inlet orifice 6 allowing through sweat, an upper layer 10 comprising an outlet orifice 22, an intermediate layer 4 located between the lower layer 3 and the upper layer 10, with the microfluidic electrochemical device 100 being formed in the thickness of the isolating support 4 forming an intermediate layer 4 extending parallel to the lower layer 3.

The microfluidic electrochemical device 100 comprises a microfluidic channel 11 that is connected to the inlet orifice 6 at a first end and is connected to the outlet orifice 22 at a second end. Thus, the flow of sweat 9 from the skin 2 of the subject is conveyed into the microfluidic channel 11, which guides the sweat from the inlet orifice 6 to the outlet orifice 22 by capillary action.

The microfluidic channel 11 is provided with an electrochemical cell 14 described hereafter, shown in FIG. 7 or FIG. 8. The electrochemical cell 14 is disposed on the inner face of the upper layer 10 closing the microfluidic channel 11 from above so as to be located in the internal space of the microfluidic channel 11.

In terms of dimensions, the diameter of the inlet orifice 6 is a few millimetres, the length of the microfluidic channel 11 ranges between 0.5 cm and 5 cm and its width ranges between 20 μm and 1,000 μm, the thickness of the intermediate layer 4 ranges between 10 μm and 500 μm, the width of the layers 3, 4, 10 of the multilayer structure ranges between 1 cm and 5 cm and the length of said layers ranges between 2 cm and 10 cm.

For example, the inlet orifice 6 has a diameter of 5 mm, the microfluidic channel 11 has a length of 3 cm and a width of 200 μm, the intermediate layer 4 has a thickness of 150 μm, the layers 3, 4, 10 of the multilayer structure have a width of 3 cm and a length of 9 cm.

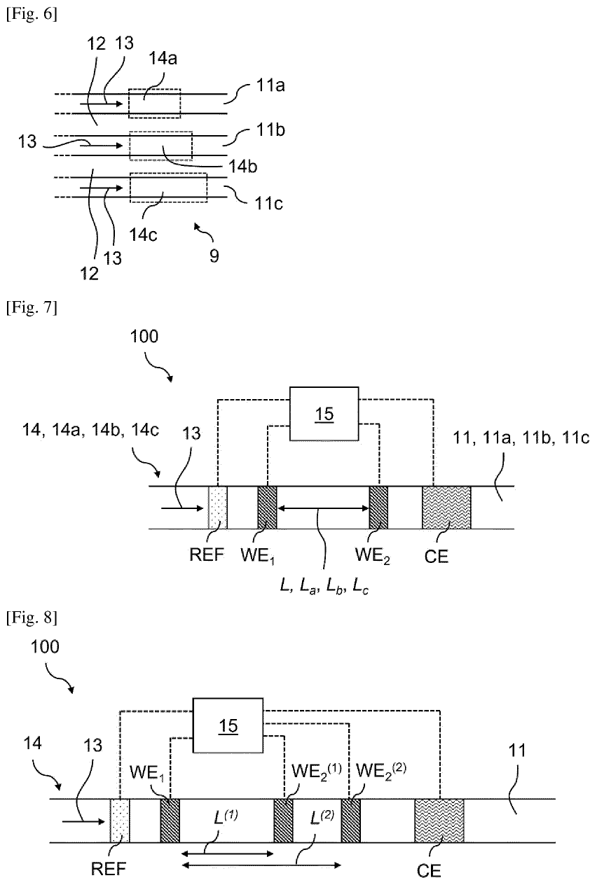

According to a second embodiment, with reference to FIGS. 5 and 6, the apparatus 1 comprises a main channel 23 dividing, in the flow direction of the flow of sweat 9, into one or more microfluidic channels, in this case three parallelepiped microfluidic channels 11a, 11b, 11c, parallel to each other, formed in the thickness of the isolating support 6 and separated by partitions 12. Each microfluidic channel 11a, 11b, 11c is thus respectively separated from the other microfluidic channels 11a, 11b, 11c within which the sweat can circulate independently. The number of microfluidic channels can be higher or lower than that shown in FIGS. 5 and 6.

In terms of dimensioning, the microfluidic channels 11a, 11b, 11c preferably have a height ranging between 10 μm and 500 μm, a width ranging between 20 μm and 1,000 μm, and a length ranging between 0.5 cm and 5 cm. The microfluidic channels 11a, 11b, 11c have a constant cross-sectional surface area Sₐ, S_{b}, S_{c}. For the sake of simplicity, but without loss of generality, the cross-sections of the microfluidic channels 11a, 11b, 11c are assumed to have the same surface area S, i.e., Sₐ=S_{b}=S_{c}=S.

The sweat perspired by the subject 2 in the investigation zone 8 is collected by the part of the collection element in contact with the skin 2 of the subject and is then transferred by capillary action to the main channel 23 in order to independently circulate in the microfluidic channels 11a, 11b, 11c of the microfluidic electrochemical device 100. The arrows 13 illustrate the flow direction of the sweat in the microfluidic channels 11a, 11b, 11c. Each microfluidic channel 11a, 11b, 11c is respectively provided with an electrochemical cell 14a, 14b, 14c, indicated in FIG. 6 and shown in detail in FIG. 7 or FIG. 8. Preferably, the microfluidic channels 11a, 11b, 11c lead to an outlet reservoir (not shown) connected to the outlet orifice 22. The outlet reservoir retains the sweat to prevent it from coming back into contact with the skin 2.

With reference to FIG. 7, the electrochemical cell 14, 14a, 14b, 14c is based on a four-electrode configuration. More specifically, the electrochemical cell 14, 14a, 14b, 14c comprises a pair of independent working electrodes WE₁ and WE₂, a counter electrode CE and a reference electrode REF, disposed in the microfluidic channel 11, 11a, 11b, 11c. The electrodes WE₁, WE₂, CE and REF are produced in the form of strips of microelectrodes parallel to each other, perpendicular to the flow direction of the sweat in the microfluidic channel 11, 11a, 11b, 11c, and are implanted by microfabrication, for example, by Chemical Vapour Deposition (CVD) and/or by lithography. The length of the microelectrode strips thus corresponds to the width of the microfluidic channel 11, 11a, 11b, 11c.

The working electrodes WE₁ and WE₂ can be produced in the form of microstrips made of platinum or platinized platinum, also known as platinum black, with a nanometric thickness, for example, of the order of a few tens of nanometres to a hundred nanometres, typically 200 nm. The working electrodes WE₁ and WE₂ are spaced apart by an inter-electrode distance L, Lₐ, L_{b}, L_{c} in the flow direction of the sweat in the microfluidic channel 11, 11a, 11b, 11c. As will be explained hereafter, the inter-electrode distance L, Lₐ, L_{b}, L_{c} varies as a function of the considered microfluidic channel 11, 11a, 11b, 11c. The first working electrode is denoted using reference sign WE₁ and the second working electrode is denoted using reference sign WE₂. By convention, the first working electrode WE₁ is located upstream of the second working electrode WE₂ relative to the flow direction of the sweat of the subject 2 in the microfluidic channel 11, 11a, 11b, 11c.

The reference electrode REF, which is produced, for example, in the form of a strip of Ag/AgCl reference microelectrode with a nanometric thickness, for example, 500 nm, is located upstream of the pair of working electrodes WE₁ and WE₂ in order to maintain the stability of the reference electrode potential over time.

The counter-electrode CE, which is produced, for example, in the form of a microstrip, for example, made of platinum, which may or may not be platinized, with a nanometric thickness, for example, of the order of a few tens of nanometres to a few hundred nanometres, typically 100 nm, is located downstream of the pair of working electrodes WE₁ and WE₂ and, therefore, of the reference electrode REF so that the chemical species generated on its surface disrupt neither the working electrodes WE₁ and WE₂ nor the reference electrode REF. Advantageously, the surface of the counter-electrode CE is two to three times larger than those of the other electrodes.

Advantageously, the microelectrodes produced in the form of microstrips are all deposited on a sub-nanometric adhesion layer (not shown), for example, made of titanium or chromium or the like depending on the nature of the isolating support 4, in order to properly adhere the microstrips on the isolating support 4.

The microfluidic electrochemical device 100 also comprises an electrochemical amperometry measurement system 15. Each of the electrodes WE₁, WE₂, CE and REF is connected to the electrochemical amperometry measurement system 15 by means of electrical contacts (not shown) electrically isolated from the sweat of the subject.

The electrochemical amperometry measurement system 15 comprises, for example, a potentiostat or a multipotentiostat (not shown) configured to control one or all or some of the electrochemical cells 14, 14a, 14b and 14c. More specifically, the electrochemical amperometry measurement system 15 is configured to bias the first and second working electrodes WE₁ and WE₂ of an electrochemical cell 14, 14a, 14b, 14c, respectively, at the first and second electrode potentials E₁ and E₂ so as to generate an oxidation reaction or a reduction reaction in the sweat that is associated with water H₂O₍ₗ₎ (examples 1 and 2) or even the reaction of a chemical species of a redox couple associated with water H₂O₍ₗ₎, in particular dioxygen O_{2(aq)} previously dissolved in the sweat (example 3).

The average volumetric flow rate Q of sweat circulating in a microfluidic channel 11, 11a, 11b or 11c is determined on the basis of the principle of the “time of flight” technique, i.e., based on measuring the time required for an electroactive chemical species, detected by amperometry, to travel the inter-electrode distance L, Lₐ, L_{b}, L_{c} between the first and second working electrodes WE₁ and WE₂, and the volume of the microfluidic channel 11, 11a, 11b, 11c delimited by the planes perpendicular to the plane in which the working electrodes WE₁, WE₂ are crimped and located at the most upstream limit of each.

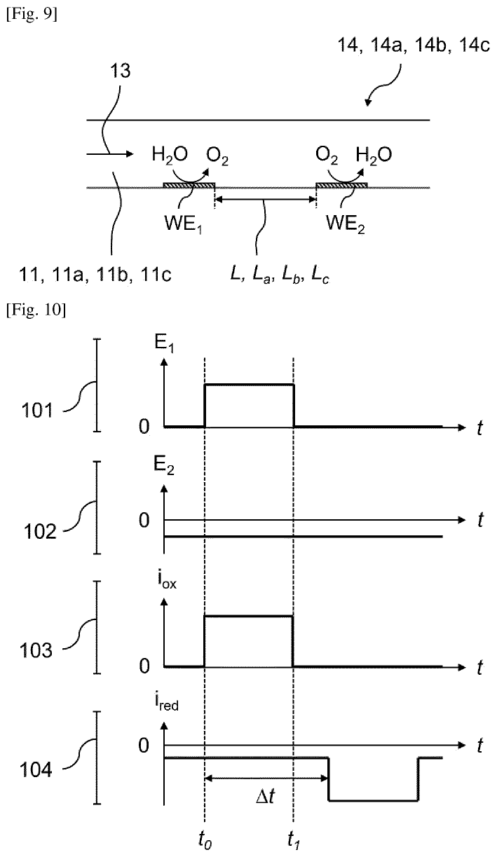

In the following three examples, the working electrodes WE₁ and WE₂ are respectively biased according to the methods illustrated in FIGS. 9 and 10 (example 1), 11 and 12 (example 2), and 13 and 14 (example 3). These methods comprise several steps that are explained in more detail for each of the particular examples described hereafter. The graphs 101, 121 and 141 show the first electrode potential E₁ applied to the first working electrode WE₁ as a function of time t. The graphs 102, 122 and 142 show the second electrode potential E₂ applied to the second working electrode WE₂ as a function of time t. When the first and second electrode potentials E₁ and E₂ assume the value of “0” on graphs 101, 102, 121, 122, 141 and 142, this corresponds to the disconnection of the corresponding working electrode WE₁ or WE₂ (open circuit) or even to the application of a potential close to or equal to the equilibrium potential. The graphs 103, 123 and 143 show the intensity of the Faradic current measured at the first working electrode WE₁ as a function of time t. Finally, the graphs 104, 124 and 144 show the Faradic current intensity measured at the second working electrode WE₂ as a function of time t.

The values of the electrode potentials E₁ and E₂ that are provided by way of an example are provided in volts with respect to the standard hydrogen electrode (V/SHE). By convention, the anodic intensity of the Faradic current assumes positive values, while the cathodic intensity of the Faradic current assumes negative values.

### Example 1

In a first example, with reference to FIG. 9, the difference in electrode potentials between the first working electrode WE₁ and the counter-electrode CE is set so that the first working electrode WE₁ is biased at a first electrode potential E₁, typically 1.6 V/SHE, allowing water H₂O₍ₗ₎ to oxidize to dioxygen O_{2(aq)} according to the following redox half-equation:

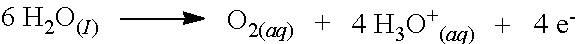

Correspondingly, the difference in electrode potentials between the second working electrode WE₂ and the counter-electrode CE is set so that the second working electrode WE₂ is biased at a second electrode potential E₂, typically −0.3 V/SHE, allowing the oxygen O_{2(aq)} produced on the surface of the working electrode WE₁ that is dissolved in the sweat to be reduced to water H₂O₍ₗ₎ according to the following redox half-equation:

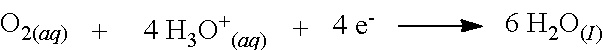

With reference to FIG. 10, the method for biasing the first and second working electrodes WE₁ and WE₂ comprises two successive steps over time t.

During a first step, before an instant t₀ (i.e., for t<t₀) the first electrode potential E₁ applied to the first working electrode WE₁ can be close to the initial equilibrium potential or the first working electrode WE₁ even can be disconnected. In this latter hypothesis, illustrated in graph 101, the first electrode potential E₁ conventionally assumes the value of zero “0”. The detected anodic intensity iₒₓ is zero, as shown in graph 103.

Δt the same time, the second electrode potential E₂ applied to the second working electrode WE₂, illustrated in graph 102, is set to a value that is lower than the initial equilibrium potential and that is sufficient to reduce the dissolved oxygen O_{2(aq)}. The cathodic intensity i_{red} is proportional to the concentration of dioxygen O_{2(aq)} previously dissolved in the sweat, as illustrated in graph 104; if this initial concentration is zero, the cathodic intensity i_{red} is zero.

During a second step starting from the instant to, the first electrode potential E₁ applied to the first working electrode WE₁, illustrated in graph 101, is fixed on the oxidation wave of the water H₂O₍ₗ₎ so as to initiate the production of an appreciable amount of dioxygen O_{2(aq)}, in other words, so that the total concentration of dioxygen O_{2(aq)} in the vicinity of the surface of the first working electrode WE₁ is much greater than the concentration of dioxygen O_{2(aq)} previously dissolved in the sweat. The anodic intensity iₒₓ, which is a quantity that represents the amount of oxygen O_{2(aq)} generated on the surface of the first working electrode WE₁, is thus set by the first electrode potential E₁ applied to the first working electrode WE₁. The anodic intensity iₒₓ is constant over time as soon as the capacitive current linked to the potential jump is cancelled because the redox reaction contemplated at the first working electrode WE₁ is not limited by the conveyance of matter since the reactant is water H₂O₍ₗ₎ The capacitive currents associated with potential switching are not shown in the diagrams, which only consider Faradic currents.

As soon as the first working electrode WE₁ is biased at the instant to, a gradient in the concentration of dissolved oxygen O_{2(aq)} is created in the vicinity of the first working electrode WE₁. The anodic intensity iₒₓ measured at the first working electrode WE₁ increases due to the oxidation of the water H₂O₍ₗ₎ In graph 103, the increase in anodic intensity iₒₓ is schematically shown by a step or a Heaviside function. The dioxygen O_{2(aq)} concentration gradient forms a concentration front driven by convection downstream of the first working electrode WE₁ under the effect of the flow of sweat.

Δt the same time, the second working electrode WE₂ remains biased at a constant second electrode potential E₂. The second working electrode WE₂ continuously records the cathodic intensity i_{red} of the Faradic current generated by the reduction of dioxygen O_{2(aq)} to water H₂O₍ₗ₎. Thus, at the instant t₀+Δt, when the dioxygen O_{2(aq)} concentration front generated on the surface of the first working electrode WE₁ passes above the surface of the second working electrode WE₂, the detected cathodic intensity i_{red} decreases (by relative value) due to the reduction of oxygen O_{2(aq)} to water H₂O₍ₗ₎ which is indicated in graph 104. The duration Δt corresponds to the time requi_{red} for the dioxygen O_{2(aq)} front generated at the first working electrode WE₁ to travel to the second working electrode WE₂ under the effect of the flow of sweat through the microfluidic channel 11, 11a, 11b or 11c at the flow speed V.

The second step ends at an instant t₁ after the instant t₀, from which the first working electrode WE₁ is again biased at a first electrode potential E₁ close to the initial equilibrium potential, or is even disconnected, as illustrated in graph 101. The first working electrode WE₁ can be subsequently rebiased so that the method can be repeated as often as necessary in order to determine the average volume flow rate Q at successive instants that are more or less close together.

The method described herein is simple and easy to industrialize, as it requires no moving parts and no assumptions concerning the hydrodynamic regime of the flow of sweat in the microfluidic channel. The solution is by no means associated with determining the concentration of chemical species generated or contained in the sweat, but only to a response time Δt between the amperometric signals of a pair of working electrodes WE₁ and WE₂. In particular, the selection of the oxidation reactions of water H₂O₍ₗ₎ and of the reduction of dioxygen O_{2(aq)} allows the amplitude of the detected amperometric signals to be guided to each of the working electrodes WE₁, WE₂ so as to maintain a sufficient signal-to-noise ratio to allow easy detection of the variation in the amperometric signals.

### Example 2

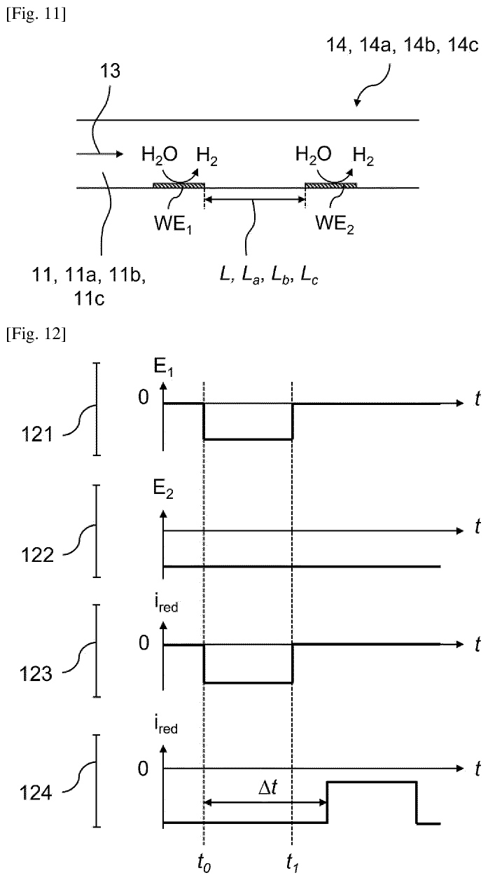

In a second example, with reference to FIG. 11, the difference in electrode potentials between the first working electrode WE₁ and the counter-electrode CE is set so that the first working electrode WE₁ is biased at a first electrode potential E₁, typically −0.8 V/SHE, allowing the reduction of water H₂O₍ₗ₎ to dihydrogen H_{2(aq)} according to the following redox half-equation:

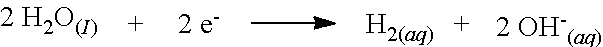

Correspondingly, the difference in electrode potentials between the second working electrode WE₂ and the counter-electrode CE is set so that the second working electrode WE₂ is biased at a second electrode potential E₂ that is equal to the first electrode potential E₁. Like the first working electrode WE₁, the second working electrode WE₂ is thus configured to reduce the water H₂O₍ₗ₎ in the sweat to dihydrogen H_{2(aq)}.

With reference to FIG. 12, the method for biasing the first and second working electrodes WE₁ and WE₂ comprises two successive steps over time t.

During a first step, before an instant t₀, i.e., for t<t₀, the first electrode potential E₁ applied to the first working electrode WE₁ can be close to the initial equilibrium potential or the first working electrode WE₁ even can be disconnected. In this latter hypothesis, illustrated in graph 121, the first electrode potential E₁ conventionally assumes the value of zero “0”, such that the detected cathodic intensity i_{red} is zero, as shown in graph 123.

Δt the same time, the second electrode potential E₂ applied to the second working electrode WE₂, shown in graph 112, is lower than the initial equilibrium potential to reduce the water in the sweat to dihydrogen H_{2(aq)}. The cathodic intensity i_{red} is constant, as illustrated in graph 124.

During a second step starting from the instant to, the first electrode potential E₁ applied to the first working electrode WE₁, illustrated in graph 111, is lower than the initial equilibrium potential so as to initiate the reduction of the water in the sweat to dihydrogen H_{2(aq)}. The cathodic intensity i_{red} detected at the first working electrode WE₁ decreases (by relative value) due to the increase in pH imposed by the reduction of water H₂O₍ₗ₎ at the first working electrode WE₁. In graph 123, the decrease in the growth of the cathodic intensity i_{red} is schematically shown by a step or a Heaviside function. The portion of the hydrolyzed sweat is carried by convection downstream of the first working electrode WE₁ under the effect of the flow.

Δt the same time, the second working electrode WE₂ remains biased at a constant second electrode potential E₂. The second working electrode WE₂ continuously records the cathodic intensity i_{red} of the faradic current generated by reducing the water in the sweat to dihydrogen H_{2(aq)}. Thus, at the instant t₀+Δt, when the flow of partially hydrolyzed sweat passes above the surface of the second working electrode WE₂, the detected cathodic intensity i_{red} increases (by relative value), i.e., since the concentration of hydronium ions H₃O⁺_{(aq)} is lower than upstream of the first working electrode WE₁, which is shown in graph 114. The duration Δt corresponds to the time requi_{red} for the hydronium ion-depleted H₃O⁺_{(aq)} sweat flow front generated at the first working electrode WE₁ to travel to the second working electrode WE₂ under the effect of the flow of sweat through the microfluidic channel 11, 11a, 11b, 11c at the flow speed V.

The volume of sweat that has experienced an increase in pH due to the action of the first electrode travels from the first working electrode WE₁ to the second working electrode WE₂ under the effect of the flow of sweat in the microfluidic channel 11, 11a, 11b, 11c at the flow speed V.

The second step ends at an instant t₁ after the instant t₀, from which the first working electrode WE₁ is again biased at a first electrode potential E₁ close to the initial equilibrium potential, or is even disconnected as illustrated in graph 121. The first working electrode WE₁ can be subsequently rebiased so that the method can be repeated as often as necessary in order to determine the average volume flow rate Q at close successive instants.

The method described herein is simple and easy to industrialize, as it requires no moving parts and no assumptions concerning the hydrodynamic regime. The solution is by no means associated with determining the concentration of chemical species generated or contained in the sweat, but only to a response time Δt between the variations in the amperometric signals of the pair of working electrodes WE₁ and WE₂.

### Example 3

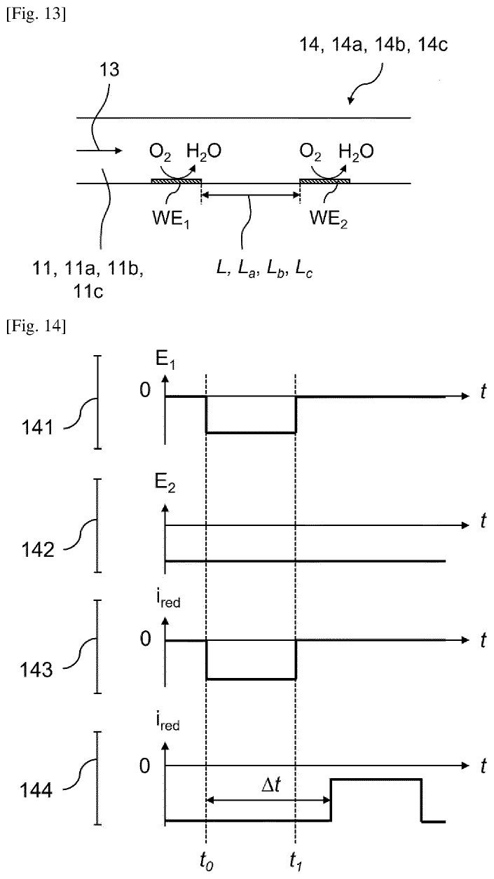

In a third example, with reference to FIG. 13, the difference in electrode potentials between the first working electrode WE₁ and the counter-electrode CE is set so that the first working electrode WE₁ is biased at a first electrode potential E₁, typically −0.3 V/SHE, allowing only the oxygen O_{2(aq)} initially dissolved in the examined aqueous solution, when it contains said solution, to be reduced to water H₂O₍ₗ₎ according to the following redox half-equation:

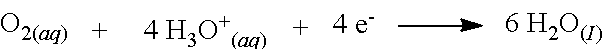

Correspondingly, the difference in electrode potentials between the second working electrode WE₂ and the counter-electrode CE is set so that the second working electrode WE₂ is biased at a second electrode potential E₂ that is equal to the first electrode potential E₁. Like the first working electrode WE₁, the second working electrode WE₂ is thus configured to reduce the fraction of oxygen O_{2(aq)} dissolved in the sweat to water H₂O₍ₗ₎ that has not been reduced at the first working electrode WE₁.

With reference to FIG. 14, the method for biasing the first and second working electrodes WE₁ and WE₂ comprises two successive steps over time t.

During a first step, before an instant t₀, i.e., for t <to, the first electrode potential E₁ applied to the first working electrode WE₁ can be close to the initial equilibrium potential or the first working electrode WE₁ even can be disconnected. In this latter hypothesis, illustrated in graph 141, the first electrode potential E₁ conventionally assumes the value of zero “0”, such that the detected cathodic intensity i_{red} is zero, as shown in graph 143.

Δt the same time, the second electrode potential E₂ applied to the second working electrode WE₂, shown in graph 142, is lower than the initial equilibrium potential for reducing the dioxygen O_{2(aq)} to water H₂O₍ₗ₎ The cathodic intensity i_{red} is constant since it is proportional to the concentration of dioxygen O_{2(aq)} previously dissolved in the sweat, as illustrated in graph 144.

During a second step starting from the instant t₀, the first electrode potential E₁ applied to the first working electrode WE₁, illustrated in graph 141, is lower than the initial equilibrium potential so as to initiate the reduction of all or some of the dioxygen O_{2(aq)} dissolved in the sweat. The cathodic intensity i_{red} detected at the first working electrode WE₁ decreases (by relative value) as a result of the reduction of oxygen O_{2(aq)} to water H₂O₍ₗ₎ In graph 143, the decrease in the growth of the cathodic intensity i_{red} is schematically shown by a step or a Heaviside function. The hydrolyzed portion of the dissolved dioxygen-depleted O_{2(aq)} sweat is carried by convection downstream of the first working electrode WE₁ under the effect of the flow.

Δt the same time, the second working electrode WE₂ remains biased at a constant second electrode potential E₂. The second working electrode WE₂ continuously records the cathodic intensity i_{red} of the Faradic current generated by the reduction of dioxygen O_{2(aq)} to water H₂O₍ₗ₎. Thus, at the instant t₀+Δt, when the flow of dioxygen-depleted O_{2(aq)} sweat passes above the surface of the second working electrode WE₂, the detected cathodic intensity i_{red} increases (by relative value), i.e., it approaches the zero value, since the concentration of dioxygen O_{2(aq)} dissolved in the sweat is zero or, at least, lower than upstream of the first working electrode WE₁, which is shown in graph 144. The duration Δt corresponds to the time requi_{red} for the flow of dissolved dioxygen-depleted O_{2(aq)} sweat to travel from the first working electrode WE₁ to the second working electrode WE₂ under the effect of the flow of sweat through the microfluidic channel 11, 11a, 11b or 11c at the flow speed V.

The second step ends at an instant t1 after the instant t₀, from which the first working electrode WE₁ is again biased at a first electrode potential E₁ close to the initial equilibrium potential, or is even disconnected, as illustrated in graph 141. The first working electrode WE₁ can be subsequently rebiased so that the method can be repeated as often as necessary in order to determine the average volume flow rate Q at close successive instants.

The method described herein is simple and easy to industrialize, as it requires no moving parts and no assumptions concerning the hydrodynamic regime. The solution is by no means associated with determining the concentration of chemical species generated or contained in the sweat, but only to a response time Δt between the variations in the amperometric signals of the pair of working electrodes WE₁ and WE₂.

In the three examples described above, the flow speed V and the volume flow rate Q of sweat flowing through a microfluidic channel 11, 11a, 11b or 11c of rectangular parallelepiped shape with a constant cross-sectional surface area S in the flow direction 13 can be determined based on the inter-electrode distance L, Lₐ, L_{b}, L_{c} separating the working electrodes WE₁ and WE₂, and based on the duration Δt characteristic of the delay in the response of the second working electrode WE₂, monitored by chronoamperometry, relative to the instantaneous response of the first working electrode WE₁.

Subject to the reservations that will be described hereafter, the average linear flow speed V and the average volumetric flow rate Q of the flow of sweat circulating in a microfluidic channel, for example, the microfluidic channel referenced 11 and for which the inter-electrode distance is referenced L, can be estimated according to the following equations:

$$V = L / Δ t \\ Q = S \times V = L \times S / Δ t$$

With the same reservations, these equations are also valid, respectively, in the microfluidic channels 11a, 11b, 11c by substituting the inter-electrode distance L with the inter-electrode distance Lₐ, L_{b}, L_{c}.

Within the context of the contemplated dynamic applications, for temporally monitoring the physiological state of the subject, it is beneficial for the apparatus 1, in order to determine the quantitative sweating parameter of a subject, to measure the value of the volumetric flow rate Q of sweat at successive instants in close succession, consistent with the expected sweating rate, for example, once a minute. Integrating any temporal variations in the volume flow rate Q of sweat then allows the value of the total flow of sweat perspired by the subject to be determined over a given time range t.

The quantitative sweating parameter can be a sweating rate that is determined based on the total volume of sweat perspired by the subject over a given time range t, in relation to the surface area of the investigation zone 8.

The inter-electrode distance L, Lₐ, L_{b}, L_{c} separating the working electrodes WE₁ and WE₂ is selected so to be small enough for changes in the physiological response of the subject to be negligible over the duration Δt and large enough to allow a decoupled operating regime for the working electrodes WE₁ and WE₂ in the one or each microfluidic channel 11, 11a, 11b, 11c where the volume flow rate Q is measured.

Indeed, depending on the average linear flow speed V of the sweat in the microfluidic channel 11, 11a, 11b or 11c, the concentration gradient created in the vicinity of the first working electrode, by generating electroactive chemical species (example 1) or by depleting electroactive chemical species already present in the sweat (examples 2 and 3), may or may not become homogeneous over the height of the microfluidic channel 11, 11a, 11b or 11c after being carried over the inter-electrode distance L, Lₐ, L_{b}, L_{c}. In particular, when, given the linear flow speed V of the sweat, the concentration gradient does not have time to dissipate over the height of the microfluidic channel 11a, 11b or 11c before reaching the second working electrode WE₂, the operation of the two working electrodes WE₁ and WE₂ is linked. This coupling regime limits the temporal resolution of the amperometric signals, which disrupts the measurements of the volume flow rate Q of the sweat circulating in the microfluidic channel 11a, 11b or 11c. This obstacle is easily avoided by adjusting the relative values of the inter-electrode distance L, Lₐ, L_{b}, L_{c} and the duration t₁-t₀ to the expected values of the average linear flow speed V.

According to a first embodiment, shown in FIG. 6, pairs of first and second working electrodes WE₁ and WE₂ separated by different inter-electrode distances Lₐ, L_{b}, L_{c} can be used in separate parallel microfluidic channels 11a, 11b, 11c. Preferably, the inter-electrode distance Lₐ, L_{b}, L_{c} separating the working electrodes WE₁ and WE₂ differs as a function of the considered microfluidic channel 11a, 11b or 11c, for example, such that Lₐ<L_{b}<L_{c}.

As a variant, according to a second embodiment illustrated in FIG. 8, an array of second working electrodes, in this case a first second working electrode WE₂⁽¹⁾ and a second second working electrode WE₂⁽²⁾, can be implemented in the same microfluidic channel 11, 11a, 11b, 11c. According to some embodiments, not shown, the array of second working electrodes can include more than two second working electrodes. The remainder of the description will be limited to describing the array of second working electrodes implemented in the microfluidic channel referenced 11. Such an array also could be implemented in the microfluidic channels 11a, 11b, 11c.

In the microfluidic channel 11, each second working electrode WE₂⁽¹⁾ , WE₂⁽²⁾ is respectively disposed at a different inter-electrode distance L⁽¹⁾ , L⁽²⁾ from the first working electrode WE₁. The working electrodes WE₁, WE₂⁽¹⁾ , WE₂⁽²⁾ are electronically switchable. The duration t1-to is electronically adjusted by feeding back the average linear flow speed value V measured over the previous measurement instants.

The volume flow rate Q thus can be determined over a wide range of values, since the volume flow rate Q can be measured in each of the microfluidic channels 11, 11a, 11b and 11c or in several of them, while retaining only the volume flow rate Q measurements consistent with the inter-electrode distances Lₐ, L_{b}, L_{c}, L⁽¹⁾ , L⁽²⁾ . Advantageously, the inter-electrode distances Lₐ, Lb and L_{c}, L⁽¹⁾ , L⁽²⁾ are of the order of a millimetre.

The methods for measuring the volumetric flow rate Q of sweat described above can be implemented automatically using an electronic processing device 16, preferably integrated into the apparatus 1.

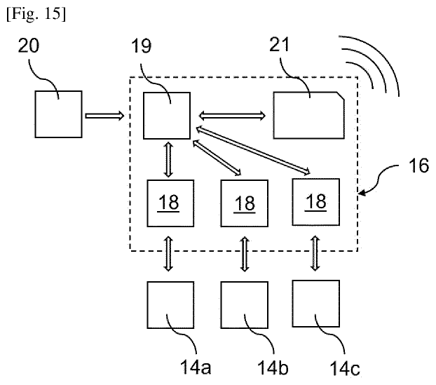

With reference to FIG. 15, an embodiment will now be described of the electronic processing device 16 that can be integrated into the apparatus 1, for example, in the form of an electronic board 17.

In the embodiment with a plurality of microfluidic channels 11a, 11b, 11c shown in FIGS. 5 and 6, the electrochemical cells 14a, 14b, 14c are connected to an analogue-to-digital converter 18, which itself powers a processor 19. The processor 19 is programmed, for example, to implement the methods for measuring the volume flow rate Q of sweat described above.

In the embodiment shown in FIG. 8, where a first working electrode WE₁ and several second working electrodes WE₂⁽¹⁾ , WE₂⁽²⁾ respectively located at different inter-electrode distances L⁽¹⁾ and L⁽²⁾ are implemented in the same microfluidic channel 11, each pair formed by the first working electrode WE₁ and one of the second working electrodes WE₂⁽¹⁾ , WE₂⁽²⁾ forms an electrochemical cell 14 connected to an analogue-to-digital converter 18, which itself powers a processor 19. The processor 19 is programmed, for example, to implement the methods for measuring the volume flow rate Q of sweat described above.

An energy source 20, for example, a battery, powers the electronic processing device 16. A wi_{red} or wireless communication module 21 also can be provided in order to send the results of the sweat volume flow rate Q measurements to a storage or post-processing apparatus.

The electronic processing device 16 optionally comprises other functional modules, for example, a gyroscopic and/or an accelerometric module for detecting the orientation and the movements of the subject 2, as well as for quantifying their level of activity, and/or a temperature sensor for measuring the temperature of the epidermis of the subject 2. Indeed, it is worthwhile knowing the skin temperature due to the correlations between the temperature and the sweating rate.

Some elements of the apparatus 1, notably the electronic processing device 16, can be produced in various forms, in a unitary or distributed manner, by means of hardware and/or software components. Hardware components that can be used include Application-Specific Integrated Circuits (ASICs) and Field-Programmable Gate Arrays (FPGAs). Software components can be written in various programming languages, for example, C, C++, Java or VHDL. This list is not exhaustive.

Although the invention has been described in conjunction with several particular embodiments, it is obvious that it is by no means limited thereto and that it includes all the technical equivalents of the means described, as well as the combinations thereof, if they fall within the scope of the invention.

The use of the verbs “comprise” and “include” and the conjugated forms thereof does not exclude the presence of elements or steps other than those set forth in a claim.

In the claims, any reference sign between brackets should not be understood to be a limitation of the claim.

1. A microfluidic electrochemical device (100) for measuring a volume flow rate (Q) of a fluid, the fluid comprising a solvent, the microfluidic electrochemical device (100) comprising:
at least one microfluidic channel (11, 11a, 11b, 11c) configured to allow the fluid to flow in a flow direction (13);
at least one electrochemical cell (14, 14a, 14b, 14c) disposed in the at least one microfluidic channel (11, 11a, 11b, 11c), the electrochemical cell (14, 14a, 14b, 14c) comprising a first working electrode (WE1) and at least one second working electrode (WE2, WE2(1), WE2(2)), with said at least one second working electrode (WE2, WE2(1), WE2(2)) being spaced apart from the first working electrode (WE1) by an inter-electrode distance (La, Lb, Lc, L(1), L(2)) in the flow direction (13), at least one counter-electrode (CE) and at least one reference electrode (REF);
an electrochemical amperometry measurement system (15) configured to bias the first working electrode (WE1) at a first electrode potential (E1) and the second working electrode (WE2, WE2(1), WE2(2)) at a second electrode potential (E2), so that each of said first and second working electrodes produces an amperometric signal by oxidation reaction or by reduction reaction of the solvent or with at least one chemical species forming a redox couple with the solvent; and
the electrochemical amperometry measurement system (15) being configured to determine the volume flow rate (Q) of the fluid in the microfluidic channel (11, 11a, 11b, 11c) based on the inter-electrode distance (La, Lb, Lc, L(1), L(2)) and a time delay (Δt) between a variation in the amperometric signal produced by the first working electrode (WE1) and a variation in the amperometric signal produced by the second working electrode (WE2, WE2(1), WE2(2)).

2. The microfluidic electrochemical device (100) as claimed in claim 1, wherein the solvent is water H2O.

3. The microfluidic electrochemical device (100) as claimed in claim 2, wherein the fluid is sweat from a human or animal subject.

4. The microfluidic electrochemical device (100) as claimed in claim 2, wherein the first electrode potential (E1) allows the oxidation of water H2O to dioxygen O2 and the second electrode potential E2 allows the reduction of the dioxygen O2 dissolved in the produced water H2O to water H2O.

5. The microfluidic electrochemical device (100) as claimed in claim 2, wherein the first electrode potential (E1) allows the reduction of water H2 O to dihydrogen H2 and the second electrode potential (E2) allows the reduction of water H2O to dihydrogen H2.

6. The microfluidic electrochemical device (100) as claimed in claim 2, wherein the first electrode potential (E1) allows the reduction of dioxygen O2 dissolved in water H2O to water H2O and the second electrode potential (E2) allows the reduction of dioxygen O2 dissolved in water H2O to water H2O.

7. The microfluidic electrochemical device (100) as claimed in claim 1, wherein the electrochemical amperometry measurement system (15) is also configured to:
during a first step, bias the first working electrode (WE1) at the first electrode potential (E1) and the second working electrode (WE2) at the second electrode potential (E2); and
during a second step, disconnect the first working electrode (WE1) or set the first electrode potential (E1) at a potential close to or equal to a zero-current equilibrium potential.

8. The microfluidic electrochemical device (100) as claimed in claim 1, further comprising an isolating support (4), said at least one microfluidic channel (11, 11a, 11b, 11c) being formed in the isolating support (4), the first working electrode (WE1) and said at least one second working electrode (WE2, WE2(1), WE2(2)) being formed by metal deposits of platinum or platinum black on said isolating support (6).

9. The microfluidic electrochemical device (100) as claimed in claim 1, wherein the counter-electrode (CE) is positioned downstream of the working electrodes (WE1, WE2, WE2(1), WE2(2)) in the flow direction (13), and wherein the reference electrode (REF) is positioned upstream of said working electrodes (WE1, WE2, WE2(1), WE2(2)) in said flow direction (13).

10. The microfluidic electrochemical device (100) as claimed in claim 1, comprising a first and a second microfluidic channel (11a, 11b), with the first, respectively, the second, electrochemical cell (14a, 14b) being disposed in the first, respectively, the second, microfluidic channel (11a, 11b), with the inter-electrode distance (La) of the first electrochemical cell (14a) being different from the inter-electrode distance (Lb) of the second electrochemical cell (14b).

11. The microfluidic electrochemical device (3) as claimed in claim 1, wherein said at least one electrochemical cell (14a) comprises two second working electrodes (WE2(1), WE2(2)) respectively separated from the first working electrode (WE1) by a first inter-electrode distance (L(1)) and by a second inter-electrode distance (L(2)), with the first inter-electrode distance (La (1)) being different from the second inter-electrode distance (L(2)).

12. The microfluidic electrochemical device (100) as claimed in claim 1, wherein the electrochemical amperometry measurement system (15) is configured to determine the volume flow rate (Q) as a function of a cross-sectional surface area(S) of said microfluidic channel (11, 11a, 11b, 11c) in the flow direction (13).

13. An apparatus (1) intended to be placed on an investigation zone (8) of an epidermis of a human or animal subject in order to measure a quantitative sweating parameter of the subject, said apparatus (1) comprising:
a structure defining a microfluidic electrochemical device (100) as claimed in claim 1, the structure comprising an inlet orifice (6) defining the investigation zone (8) and allowing through sweat from the epidermis, the at least one microfluidic channel (11, 11a, 11b, 11c) of the microfluidic electrochemical device (100) being connected to the inlet orifice (6); and
an electronic processing device (16) configured to determine the quantitative sweating parameter of said human or animal subject based on measurements of the volume flow rate (Q) of sweat carried out by the microfluidic electrochemical device (100).

14. The apparatus (1) as claimed in claim 13, wherein the quantitative sweating parameter of said human or animal subject is a sweating rate.

15. The apparatus (1) as claimed in claim 13, wherein the structure is a multi-layer structure comprising a lower layer (3) and at least one layer superimposed on the lower layer (3), with the microfluidic electrochemical device (100) extending parallel to the lower layer (3), the lower layer (3) comprising said inlet orifice (6).

16. The apparatus (1) as claimed in claim 15, wherein the multi-layer structure further comprises an upper layer (10) and at least one intermediate layer (4) located between the lower layer (3) and the upper layer (10), with the microfluidic electrochemical device (100) being formed within the thickness of the at least one intermediate layer (6).

17. The apparatus (1) as claimed in claim 16, wherein the upper layer (10) has an outlet orifice (22) passing through the upper layer (10), and wherein the at least one microfluidic channel (11, 11a, 11b, 11c) is connected to the outlet orifice (22).

18. The apparatus (1) as claimed in claim 16, wherein the first working electrode (WE1), the at least one second working electrode (WE2, WE2(1), WE2(2)), the at least one counter-electrode (CE) and the at least one reference electrode (REF) are disposed on an inner face of the upper layer (10) closing the at least one microfluidic channel (11, 11a, 11b, 11c) from above and/or are disposed on an upper face of the lower layer (3) closing said at least one microfluidic channel (11, 11a, 11b, 11c) from below.

19. The apparatus (1) as claimed in claim 13, further comprising a communication device (21) configured to transmit one or more measurement signals produced by the microfluidic electrochemical device (100).

20. The apparatus (1) as claimed in claim 13, further comprising a gyroscopic module and/or at least one accelerometer for detecting a state of activity of said human or animal subject.

21. The apparatus (1) as claimed in claim 13, further comprising a temperature sensor configured to measure the temperature of the epidermis (2) of said human or animal subject.
